(19) United States
(12) Patent Application Publication
(10) Pub. No.: US 2020/0229739 A1
(43) Pub. Date: Jul. 23, 2020

(54) Detecting Substances Using A Wearable Oral Device

(71) Applicant: Canary Health Technologies, Inc., Toronto (CA)

(72) Inventor: Raj Reddy, Burlington (CA)

(21) Appl. No.: 16/749,471

(22) Filed: Jan. 22, 2020

Related U.S. Application Data
(60) Provisional application No. 62/795,199, filed on Jan. 22, 2019.

(51) Int. Cl. A61B 5/1486; A61B 5/00; A61B 5/08; A61B 5/087; A61B 5/083

(52) U.S. Cl. CPC A61B 5/1486; A61B 5/002; A61B 5/682; A61B 5/082; A61B 5/4833; A61B 5/4866; A61B 5/087; A61B 5/0833; A61B 5/0836; A61B 5/4277

(57) ABSTRACT

Systems and methods for detecting substances using a wearable oral device are described. For example, a wearable device is described comprising a mouth guard, a sensor coupled to the mouth guard and being configured to detect chemical signals, and a transmitter coupled to the sensor and being configured to transmit the detected chemical signals to a receiver. In another example, a wearable device is described comprising a bond being configured to be removably attachable to a tooth of a user, a sensor coupled to the bond and being configured to detect chemical signals, and a transmitter coupled to the sensor and being configured to transmit the detected chemical signals to a receiving device.

### CROSS-REFERENCE TO RELATED APPLICATION(S)

This application claims priority to and the benefit of U.S. Provisional Application Patent Ser. No. 62/795,199, filed Jan. 22, 2019, the entire disclosure of which is hereby incorporated by reference.

### TECHNICAL FIELD

This invention relates to wearable devices, in particular to the detection of substances using a wearable oral device.

### BACKGROUND

Wearable sensor devices have been utilized for on-body monitoring of a wide range of relevant parameters for health, fitness, and biomedicine applications. The majority of existing wearable technologies focus on monitoring and detecting physical parameters (e.g., motion, respiration rate, etc.) or electrophysiology (e.g., ECG, EMG, etc.) as opposed to focusing on chemical markers relevant health and fitness.

### SUMMARY OF THE INVENTION

Disclosed herein are implementations of wearable devices for detecting substances orally.

In a first aspect, a wearable device comprises a mouth guard, a sensor coupled to the mouth guard, the sensor configured to detect chemical signals, and a transmitter coupled to the sensor, the transmitter configured to transmit the detected chemical signals to a receiver.

In a second aspect, a wearable device comprises a bond, the bond configured to be removably attachable to a tooth of a user, a sensor coupled to the bond, the sensor configured to detect chemical signals, and a transmitter coupled to the sensor, the transmitter configured to transmit the detected chemical signals to a receiving device.

In a third aspect, a wearable device comprises a flow path, the flow path operable to receive and pass exhaled gases or dissolved salivary compounds, the flow path configured to contact a user so as to pass exhaled gases or dissolved salivary compounds as the user breathes or saliva of the user is contacted, a sensor array, the sensor array configured to detect the exhaled gases or salivary compounds, and a transmitting device, the transmitting device configured to transmit the detected exhaled gases or salivary compounds to an external device for real-time analysis.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates an example wearable oral device in accordance with a first embodiment.

FIG. 2 illustrates an example wearable oral device in accordance with a second embodiment.

### DETAILED DESCRIPTION

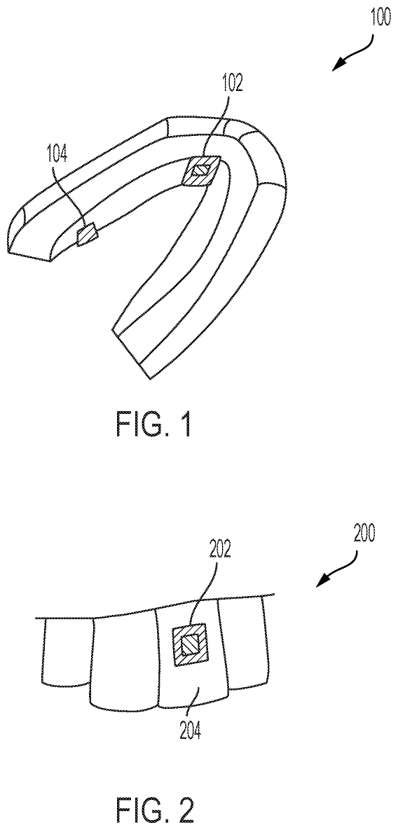

Wearable oral devices for detecting chemical substances to help detect diseases such as cancer and to help monitor medication adherence behavior of a user are provided. The present invention describes an instrumented mouthguard biosensor system (such as the device of FIG. 1) or a miniature bonded sensor device (such as the device of FIG. 2) that are capable of non-invasively monitoring nanoparticle tracking code levels used to identify medications taken by patients. The enzyme (laccase)-modified screen-printed electrode system has been integrated onto a mouthguard platform along with anatomically-miniaturized instrumentation electronics featuring a potentiostat, microcontroller, and a Bluetooth Low Energy (BLE) transceiver. Unlike RFID-based biosensing systems, which require large proximal power sources, the developed platform enables real-time wireless transmission of the sensed information to standard smartphones, laptops, and other consumer electronics for on-demand processing, diagnostics, or storage.

The mouthguard biosensor system disclosed by the present invention offers high sensitivity, selectivity, and stability towards encoded nanoparticle detection in human saliva which can be used to identify medications and track medication adherence. The mouthguard biosensor system is wireless and can monitor encoded nanoparticle levels in real-time and continuous fashion, and can be readily expanded to an array of sensors for different analytes to enable an attractive wearable monitoring system for diverse health and fitness applications.

Wearable devices have numerous physical external body applications but oral wearable devices are not yet prevalent. Saliva is a great diagnostic fluid providing an alternative to direct blood analysis via the permeation of blood constituents without any skin-piercing for blood sampling. A method and system in accordance with the present invention enables real-time monitoring of chemical markers using wearable oral devices including a mouthguard biosensor system. The mouthguard biosensor system disclosed by the present invention can be fabricated using screen-printing technology on a flexible PET (polyethylene terephthalate) substrate. Chemical modification of the printed working electrode Prussian-blue transducer can be made by crosslinking the laccase enzyme and electropolymerization. The mouthguard biosensor system can detect substances in both artificial saliva and undiluted human saliva. The mouthguard biosensor system includes an integrated with a wireless amperometric circuitry to realize a comfortable wearable device. The resulting integrated mouthguard biosensor provides real-time encoded nanoparticle measurements along with wireless data transmission. A BLE chipset is included to enable wireless connectivity to a smartwatch, smartphone, tablet, portable media player, laptop or any other BLE-enabled device. In the following sections, we will describe the design of the integrated mouthguard biosensor coupled with a miniaturized printed circuit board for wireless data collection and its attractive performance in the continuous monitoring of salivary encoded nanoparticles which identify the underlying medication.

The present invention also discloses a wearable oral device that comprises a miniature flexible sensor system that is configured to be bonded to a tooth's minutely bumpy surface. The flexible sensor can come in a variety of sizes including 2 millimeters by 2 millimeters to attach to one surface of the tooth but can also be in a cap form factor so that it can be placed around an entire tooth. The sensor includes three layers: two outer gold rings, and an inner layer of a bioresponsive material that is sensitive to selected nanoparticles and electrolytes as well as glucose. Different nanoparticle substances shift the bioresponsive material's electrical properties and cause it to transmit a different spectrum of radiofrequency waves. Together, the three layers act as an antenna, broadcasting the information to external receiving devices such as mobile devices, like smartphones or tablets.

The mouthguard biosensor system and the miniature flexible sensor system (collectively, “wearable oral devices”) can be used for monitoring medication adherence behavior of a user. The wearable oral devices can non-invasively monitor nanoparticle tracking code levels used to identify medications taken by patients and to identify other compounds in the saliva that are related to disease and health conditions.

The wearable oral devices described herein include organically or inorganically functionalized nanomaterials that fulfill the stringent requirements of saliva testing: the nanosize allows the implementation of very sensitive and reliable gas and chemical compound sensors, the adjustability of the chemical and physical properties allows optimal sensing of disease-specific VOC or VG patterns in the saliva, and the ease of fabrication renders production reasonably cost effective. The customized nanosensors can be embedded into bonded tooth sensors with wireless capabilities or into wirelessly enabled mouthguards.

In the present invention, the wireless amperometric circuit, paired with a Bluetooth low energy (BLE) communication system-on-chip (SoC) for miniaturized and low-power operation, is fully integrated into a novel salivary nanoparticle mouthguard biosensor system for continuous and real-time amperometric monitoring. The mouthguard biosensor system includes a mouthguard enzyme electrode that is applied for the detection of encoded nanoparticles for identifying medication, which is used for tracking medication adherence.

The wearable mouthguard saliva and breath sensors and/or bonded tooth sensors for saliva and/or breath testing provide a comprehensive detection and screening method for digestive cancers, which can affect in the entire digestive system: esophagus, stomach, small intestine, colon, rectum, anus, liver, pancreas, gallbladder and biliary system. Endogenous cancer-specific compounds such as volatile organic compounds (VOCs) that have been dissolved in the saliva are released from the cancer cells and/or metabolic processes that are associated with cancer growth whereby different cancers emit different types and/or amounts of molecules. These VOCs are transported with the blood to the alveoli of the lung from where they are exhaled as measurable odorants and chemical signals that are detected by the wearable oral devices disclosed by the present invention.

The wearable oral devices of the present invention provides for mobile monitoring of acetone levels in the breath or saliva via a bonded tooth sensor or mouthguard worn sensor. This provides valuable information on exercise programs and weight loss programs. Saliva is a great diagnostic fluid providing an alternative to direct blood analysis via the permeation of blood constituents without any skin-piercing for blood sampling and monitoring the saliva for encoded nanoparticles related to medication adherence as well as compounds related to disease using a wearable mouthguard (i.e., mouthguard biosensor system) or bonded tooth sensor (i.e., miniature flexible sensor system) provides a convenient and passive method for detecting health related issues.

The wearable oral devices disclosed by the present invention provide an apparatus for the measurement of the released nanoparticles in the saliva or breath using an oral wearable sensor to detect these release encoded nanoparticles originating from previously encoded oral medications. The apparatus can also detect other compounds which describe health and wellness from the saliva or breath to monitor health and wellness such as ketones like acetone but aldehydes such as acetaldehyde may also be detected.

In an implementation, a nanoparticle-based sensor apparatus (wearable oral device) is disclosed that is based on nanocomposites, nanotubules or nanofibers with immobilized substances upon them such as biological enzymes such as laccase or custom nanoparticles which are tuned to select for specific gases and substances that are found exhaled in the breath. In the case of aldehydes or ketones, the wearable oral device selectively detects them using laccase and or the custom nanoparticles which have a fixed porosity designed to adhere to selected exhaled gases such as ketones or aldehydes such as acetone thereby sensing the selected exhaled gas such as acetone.

The wearable oral devices can include electrochemical sensing materials like carbon nanofibers or CarbonNanoTubes or polymeric nanofibers are synthesized according to the selected gases to be detected. Nanoparticles are defined as a solid colloidal particles having size in the range from 10 to 1000 nm, which offers many benefits to larger particles such as increased surface-to-volume ratio and increased magnetic properties. In some implementations, the wearable oral devices are used to monitor the composition of inhaled gases, for example when administering gases to the patient such as anesthetics, nitric oxide, medications, and other treatments, monitoring pollutants or environmental effects, for a person respiring with the assistance of a ventilator, or for persons using breathing apparatus. Both exhaled and inhaled gases can be detected and analyzed by the method and system in accordance with the present invention.

In some implementations, the wearable oral device conducts ketone detection using a hand-held nanoparticle based bonded tooth sensor (i.e., miniature flexible sensor system) and/or wearable mouthguard biosensor system. A person could have the analyzer device bonded to their tooth using standard dental technology or wear a sensor enabled mouthguard. Exhaled airor saliva is in contact in the mouth with the wearable oral devices. Volatile organic compounds such as acetone can be adsorbed or selectively trapped at the molecular level on a nanoparticle surface which may be enabled with enzymes such as laccase, and detected and quantified by the selective electrochemical nanoparticle sensor system. Selectively permeable membranes may also be used to allow nitrogen, oxygen, and possibly carbon dioxide to exit a detector device, while concentrating volatile organics such as ketones for detection by a method in accordance with the present invention.

Data detected by the wearable oral devices may be transferred from the sensor via transmitting devices (e.g., a wireless transmitter) to other devices by direct attachment or wireless communication including but not limited to smartphones, portable computers, interactive television components (e.g. set-top box, web-TV box, cable box, satellite box, etc.), desktop computers, wireless phones, etc. The wireless transmitter can be via Bluetooth protocol radio communication, IR communication, transferable memory sticks, wires, WiFi, or other electromagnetic/electrical methods. Data may also be transferred to a remote computer or cloud computing infrastructure via a communications network such as the internet. In an implementation, the data detected by the wearable oral device is transferred to a smartphone directly via wireless transmission.

The following example illustrates how breath or saliva ketone measurements can be used in an improved weight loss program involving an exercise component. A person is equipped with an activity sensor (e.g. pedometer, accelerometer) and starts an activity routine (e.g. running on the spot). The wearable oral devices of the present invention including a nanoparticle sensor with additional ketone sensing capability is used to monitor the person's oxygen intake rate and hence metabolic rate and also to detect the attainment of a certain acetone level in the person's breath or saliva, indicating the onset of fat catabolism. The data is transferred to a smartphone and to the internet cloud securely for real-time processing and feedback back to the user. Data transfer to the smartphone may be done by IR communication, Bluetooth protocol wireless communication, direct connection or through the transfer of a memory stick. The data can be used to create a model of the person's physiological response to exercise.

Breath or saliva ketone sensing can also be used to detect the onset of the dangerous condition of ketoacidosis. In another implementation, a system for warning a person of the onset of ketoacidosis comprises a smartphone application carried by the person, a blood glucose sensor, and an oral wearable analyzer (i.e., wearable oral devices of the present invention) that functions as an indirect calorimeter and respired volatile organics detector and is in two way communication with the smartphone device using wireless communication. The oral wearable analyzer may be attached onto a smartphone directly or combined with mobile technology into a portable unitary device. Also, the ketone sensing device may be combined or be separate from the calorimeter.

The following example relates to exercise management. A person exercising carries a portable wearable oral ketone analyzer that includes a device bonded to the tooth or worn as a sensor enabled mouthguard that captures saliva or breath and a nanoparticle ketone detector disposed on one wall of the oral mouthguard. The device may be small, such as the size of a human thumb nail. The exerciser may periodically have their saliva or breath sampled through the device to determine whether they are burning fat. Alternatively, the device may prompt the user to periodically to make sure the mouth guard is worn, or may signal that analysis is required after a certain period of time has passed. Also, a separate exercise monitor may wirelessly signal the analyzer that the saliva should be analyzed after a certain set of conditions are met. The analyzer may wirelessly communicate the results back to an exercise monitor, may give a confirmation of results such as by a chime indicating fat burning, or may store the results versus time onto a non-volatile memory device after streaming from the device to a smartphone. The data can be streamed from the smartphone in real-time to the internet cloud for further analysis.

Therefore, the present invention discloses a method for encouraging exercise in a person which comprises monitoring a metabolic rate of a person during an exercise, correlating the exercise with metabolic rate, detecting the presence of organic compounds in the breath of the person, indicative of fat metabolizing processes in the person, determining the effect of exercise on fat burning, providing feedback to the person during future repetition of the exercise, in terms of the effect of the exercise on metabolic rate and fat burning whereby the person is encouraged to continue exercising by the provision of the feedback.

Implementations of the present invention can be used to detect numerous volatile organic compounds in the breath or saliva, which include ketones such as acetone, aldehydes such as acetaldehyde, hydrocarbons including alkanes such as pentane, alkenes, and fatty acids, and other compounds. Implementations of the present invention can further be used to detect nitric oxide, ammonia, carbon monoxide, carbon dioxide, and other components of exhaled breath. Respiration components produced by certain bacteria within the mouth, stomach, and intestinal tract can also be detected using embodiments of the present invention.

A wearable sensor enabled mouthguard or bonded tooth analyzer (i.e., wearable oral devices) according to the present invention can be combined with gas flow sensors so as have the capabilities of a spirometer. The improved spirometer is useful for detecting respiratory components such as nitric oxide diagnostic of asthma and other respiratory tract inflammations. The combination of respiratory component analysis and flow rate analysis is helpful in diagnosing respiration disorders.

Certain persons desire a diet low in carbohydrates and high in protein. A wirelessly oral sensor apparatus according to the present invention can be used to detect respiration or salivary components indicative of success in following such a diet. In an implementation, an oral analyzer for a person comprises: a bonded tooth sensor or wearable sensor enabled mouthguard onto which the person breathes or it is immersed in saliva; a metabolic rate meter, providing metabolic data correlated with the metabolic rate of the person; a ketone sensor, providing a ketone signal correlated with a concentration of respiratory components in exhalations or saliva of the person, wherein the respiratory components are correlated with a level of ketone bodies in the blood of the person; a display; and an electronic circuit, receiving the ketone signal and the metabolic data, and providing a visual indication of the metabolic rate and the ketone signal on the display. The metabolic rate meter can comprise a pair of ultrasonic transducers, for example using the density of exhaled air to determine oxygen and carbon dioxide concentrations in exhaled air.

The metabolic rate meter can comprise a pair of ultrasonic transducers or nanoparticle flow sensors or microelectronic flow sensors, for example using the density of exhaled air to determine oxygen and carbon dioxide concentrations in exhaled air. The metabolic rate meter may comprise a flow rate sensor, and an oxygen sensor and/or a carbon dioxide sensor. Embodiments of the ketone sensor are discussed in detail below. The ketone sensor can, for example, comprise a nanoparticle sensor mechanism or array to select for a particular exhaled compound such as ketones or acetone.

The wearable oral device can include a Bluetooth Low Energy (BLE) chipset to enable wireless connectivity to a smartwatch, smartphone, or laptop over the distance of several meters, enabling unobtrusive, real-time monitoring. The wearable oral device can further include an analog front end, programmable through an I2C interface as the onboard potentiostat, a fabricated printed circuitboard assembly (PCBA), a2.45 GHz chip antenna and impedance matched balun were employed for wireless transmission. Two batteries can be used in series as a power source, regulated for the electronics via a TPS61220 boost converter and an LM4120 low-dropout voltage regulator.

Assembly and characterization of integrated wireless mouthguard includes a wireless electronics board is integrated into the mouthguard platform. Stainless steel wires connected to the screen-printed electrode on PET substrate can be soldered to the fabricated PCB, and the electronics board together with the printed electrode can be assembled into the mouthguard using medical adhesive (Loctite).

The present invention includes an exemplary method for detecting compounds such as encoded nanoparticles related to medication adherence, ketones or other volatile organic compounds that define disease by training a Neural Network to classify an exhaled gas or saliva input. The present invention further includes a device with a flow path for exhaled gas or saliva through a nanoparticle ketone sensor attached to a wearable bonded tooth sensor connected to a smart phone device and the wireless transmission of correlated data from the mobile device to the cloud and the display of the data. The present invention further includes an exemplary method for detecting compounds, encoded nanoparticles related to medication adherence, ketones or volatile organic compounds from breath or saliva using a nanoparticle sensor and shows a plausible wearable mouth guard sensor fabrication using NanoFibers. The present invention further includes a sensor fabrication which can be NanoFibers or CarbonNanoFibers (CNF) with embedded selective NanoParticle which can be Nano Metal Oxide (MOX) selectively trapping an exhaled gas or saliva which can be acetone, volatile organic compounds or encoded nanoparticles related to medication adherence. The present invention further includes a sample wearable oral sensor for detecting compounds in the saliva or breath such as encoded nanoparticles related to medication adherence, ketone's or volatile organic compounds detection system in accordance with a preferred embodiment of the invention.

Implementations described herein detect and classify certain exhaled gases or salivary compounds from a person or mammal in a fluid medium or breath sample of a user and/or patient by a nanoparticle wearable oral sensor which transmits data to a smartphone mobile wireless device to the cloud for processing which can be by a neural network based processor or computerized system. The substances or exhaled gases of interest are detected by the system using electronic and/or electromechanical sensors. The sensors convert the detection of certain substances such as encoded nanoparticles related to medication adherence, ketones or volatile organic compounds in the exhaled breath or saliva into electrical signals which are conveyed to a pattern recognition system, such as neural network, and a result is generated.

FIG. 1 illustrates a mouthguard biosensor system 100 that includes a wireless amperometric circuit board 102 including a transmitter and a sensor 104 for detecting chemical substances. A reagent layer of the chemically modified printed carbon working electrode containing enzymes such as laccase for saliva or breath biosensor can be utilized. FIG. 2 illustrates a bonded tooth sensor 200 that includes a sensor system 202 that is bonded using dental adhesive to a user's tooth 204.

In an implementation, an exemplary method for classifying an exhaled gas or saliva is disclosed. The method starts with training of a neural network, for example, using known gases through a nanoparticle-based sensor. Once the neural network is trained, it is deployed. The deployed system receives one or more selected exhaled gases or saliva using a sensor or sensor group. The received exhaled gases are processed using the neural network or computerized system which, in a preferred embodiment, is an artificial neural network and one or more results are generated. The results provide identification of exhaled gases or dissolved compounds in the saliva based on received exhaled gases, or vapors or dissolved compounds in the saliva and by identifying the unique electronic sensor derived signal pattern of the exhaled gases that are correlated with the underlying substance. These results are provided to an operator in substantially real-time.

As used herein real-time refers to an event or a sequence of steps, such as are executed by a processor that are perceivable by a user or observer at substantially the same time that the event is occurring or that the steps are being performed. By way of example, if the neural network receives an exhaled gas or saliva, the system produces a result at substantially the same time that the exhaled gas or saliva was sensed. This real-time processing can input to the neural network and further associated with the processing of data by the have some time delay associated with converting sensed exhaled gas or saliva to electrical signals for neural network; however, any such delay is less than 1 minute and typically no more than a few seconds.

In another implementation, an electronic exhaled gas sensing apparatus is useful for detecting exhaled gases or saliva substances which can be ketones such as acetone, encoded nanoparticles related to medication adherence or volatile organic compounds. For example, this embodiment can be used for real-time site assessment and monitoring activities associated with diet and weightloss as well as monitoring and detection of ketones in diabetes. Afield measurement system is disclosed that is capable of detecting and classifying exhaled gases such as ketones such as acetone associated within a breath sample or saliva of a user and/or patient who is on a diet or weight control program or who is diabetic. A wearable dental guard piece which is connected to a sensing instrument module [electronic wearable oral sensor device] and linked wirelessly to a neural network collects a breath or saliva sample of patient or user which detects and displays the unique fingerprint or exhaled gas profile of that substance or gas the sensing device which can be wirelessly linked to a smart phone wireless platform to send data to the cloud wirelessly for assessment.

In another implementation, an electronic exhaled gas sensing apparatus is disclosed that includes a plausible sensor using nanomaterials which can be nanofibers is useful for detecting exhaled gases substances which can be ketones such as acetone. The sensor includes a working electrode and a counter electrode and a reference electrode. In more detail, the exhaled gas sensor includes counter electrode which can be made from a conducting paint which can be carbon paint and a working electrode which can be made from a conducting paint which can be connected to a bed of carbon nanofibers or carbon nanofibers with carbon nanotubules which can be multi-walled and a reference electrode which can be made from a conducting paint such as a silver (Ag) material. The electrode cross section can be fabricated from a bed of nanofibers which can be embedded with sensing enhancing nanoparticles for purposes such as selecting specific gas electrical fingerprint electrical signal patterns. An embodiment of a sensor fabrication comprises NanoFibers or CarbonNanoFibers (CNF) with embedded selective NanoParticle which can be Nano Metal Oxide (MOX) selectively trapping an exhaled gas in a CNF/MOX matrix which exhaled gas can be acetone which produces a complex whereby the CNF/MOX matrix is embedded with the trapped exhaled gas which can be acetone leads to a unique electrical fingerprint signal for the trapped gas or dissolved salivary compounds which can be used for identification purposes.

In another implementation, an electronic exhaled gas sensing apparatus attached to a smartphone is disclosed which includes a plausible sensor which can be NanoFibers or CarbonNanoFibers (CNF) with embedded selective NanoParticle which can be Nano Metal Oxide (MOX) selectively trapping an exhaled gas or salivary compounds which can be Acetone, encoded nanoparticles related to medication adherence or volatile organic compounds related to disease which are used for identifying, quantifying and classifying selected exhaled gases or dissolved compounds in the saliva. The device can allow users to passively sample exhale gas or saliva through a wearable oral sensor which causes the gas to flow through and over an exhaled gas sensor such as described herein which can then identify and quantify the gas an unique electrical signal fingerprint which is sent through the smart phones computerized wireless system to the internet cloud which is then processed through the clouds computerized identification system which can be a neural network and the processed identified exhaled gas signal is then returned to the mobile smart phone device or computerized system display as a visual display of the identified exhaled gas.

As described herein, the artificial neural network-based breath sensor system is capable of being trained to detect substantially any identifiable exhaled or inhaled gas or dissolved compounds in the saliva. Implementations of the invention are therefore applicable to essentially any industry or application where automated detection and classification of exhaled gases or inhaled anesthetic gas types or correlated gases or dissolved salivary compounds is desired.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures.

The claims should not be read as limited to the described order or element unless stated to that effect. Therefore, all embodiments that come within the scope and spirit of the following claims and equivalents thereto are claimed as the invention.

What is claimed:

1. A wearable device, comprising:
a mouth guard;
a sensor coupled to the mouth guard, the sensor configured to detect chemical signals; and
a transmitter coupled to the sensor, the transmitter configured to transmit the detected chemical signals to a receiver.

2. The wearable device of claim 1, wherein the chemical signals include nanoparticle respiratory signals.

3. The wearable device of claim 1, wherein the chemical signals include nanoparticle saliva signals.

4. The wearable device of claim 1, wherein the receiver is an external device.

5. The wearable device of claim 4, wherein the external device is a smartphone.

6. The wearable device of claim 1, wherein the receiver processes the detected chemical signals to provide real-time analytical data.

7. The wearable device of claim 1, wherein the sensor is a metabolic rate meter.

8. The wearable device of claim 1, wherein the sensor is a nanoparticle flow rate sensor.

9. The wearable device of claim 1, wherein the sensor a nanoparticle oxygen sensor.

10. The wearable device of claim 1, wherein the sensor is a nanoparticle carbon dioxide sensor.

11. The wearable device of claim 1, wherein the sensor is a ketone sensor.

12. The wearable device of claim 1, wherein the sensor is configured to capture exhaled breath and dissolved salivary compounds.

13. The wearable device of claim 12, wherein the sensor is configured to detect a concentration of respiratory and dissolved salivary compounds.

14. The wearable device of claim 13, wherein the concentration of respiratory and dissolved salivary compounds corresponds to encoded nanoparticles to identify medications for medication adherence.

15. The wearable device of claim 13, wherein the concentration of respiratory and dissolved salivary compounds corresponds to volatile organic compounds to identify diseases including cancer.

16. A wearable device, comprising:
a bond, the bond configured to be removably attachable to a tooth of a user;
a sensor coupled to the bond, the sensor configured to detect chemical signals; and
a transmitter coupled to the sensor, the transmitter configured to transmit the detected chemical signals to a receiving device.

17. The wearable device of claim 16, wherein the chemical signals include respiratory and salivary substances.

18. The wearable device of claim 16, wherein the receiving device includes a smartphone, a computing device, a cloud computing device, or a server.

19. The wearable device of claim 16, wherein the detected chemical signals correspond to encoded nanoparticles or volatile organic compounds for identifying medications for medication adherence or identifying diseases including cancer.

20. A wearable oral sensor analyzer, comprising:
a flow path, the flow path operable to receive and pass exhaled gases or dissolved salivary compounds, the flow path configured to contact a user so as to pass exhaled gases or dissolved salivary compounds as the user breathes or saliva of the user is contacted;
a sensor array, the sensor array configured to detect the exhaled gases or salivary compounds; and
a transmitting device, the transmitting device configured to transmit the detected exhaled gases or salivary compounds to an external device for real-time analysis.
